(19) United States
(12) Patent Application Publication
(10) Pub. No.: US 2025/0017958 A1
(43) Pub. Date: Jan. 16, 2025

(54) COMBINATORIAL THERAPEUTIC APPROACH FOR FRIEDREICH'S ATAXIA

(71) Applicant: University of Florida Research Foundation, Incorporated, Gainesville, FL (US)

(72) Inventor: Vijayendran Chandran, Gainesville, FL (US)

(21) Appl. No.: 18/707,245

(22) PCT Filed: Nov. 28, 2022
(86) PCT No.: PCT/US2022/080481

§ 371 Date: May 3, 2024

Related U.S. Application Data
(60) Provisional application No. 63/284,777, filed on Dec. 1, 2021.

(51) Int. Cl. A61K 33/26; A61K 31/145; A61K 31/353; A61P 9/06; A61P 25/28

(52) U.S. Cl. CPC A61K 33/26; A61K 31/145; A61K 31/353; A61P 9/06; A61P 25/28

(57) ABSTRACT

Described are compositions comprising two or more of quercetin, taurine, epigallocatechin gallate, and ferrous sulfate. The compositions can be used to treat Friedreich's ataxia. Pharmaceutical formulations and methods of using the compositions and pharmaceutical formulations are also described. The compositions, pharmaceutical formulations, and methods can be used to treat subjects suffering from Friedreich's ataxia or to prevent or alleviate one or more symptoms associated with Friedreich's ataxia.

### CROSS-REFERENCE TO RELATED APPLICATIONS

This Application claims the benefit of U.S. Provisional Application No. 63/284,777, filed Dec. 1, 2021, which is incorporated herein by reference.

### BACKGROUND

Friedreich's ataxia (FRDA) is the most common multisystem autosomal recessive neurodegenerative disease. About 98% of FRDA is caused by severely reduced levels of frataxin (FXN) resulting from a GAA trinucleotide repeat expansion within the first intron of the FXN gene. A larger GAA expansion correlates with residual FXN levels, earlier onset, and increased disease severity. Disease-associated, expanded alleles (fxn) contain 600 to 900 repeats on average. Heterozygous GAA expansion carriers (FXN/fxn) are not clinically affected, and the prevalence ranges from 1:60 and 1:110 among European populations. About 2% of cases of FRDA are due to other mutations, including point mutations in the FXN gene. In homozygotes, FRDA causes progressive damage to the spinal cord, peripheral nerves, and the cerebellum portion of the brain. The condition progressively impairs motor function, leading to ataxic gait, neuronal degeneration, cardiac abnormalities, and other comorbidities, ultimately resulting in early mortality. FRDA conditions tend to develop in children and teens and gradually worsens over time. Median age of death is 35 years. Currently, there is no cure for Friedreich's ataxia. Treatment focuses on minimizing symptoms and maintaining comfort and function for as long as possible.

The GAA expansion length is the major determinant of progression rate in FRDA. The GAA expansion results in FXN deficiency through an unclear molecular mechanism, resulting in the development of FRDA. FXN deficiency leads to selective dysfunction of DRG neurons, the spinocerebellar and corticospinal tracts, the dentate nuclei of the cerebellum, optic nerves, peripheral sensory nerves, cardiomyocytes, and pancreatic beta cells, whereas other tissues are spared. FXN is a nuclear-encoded mitochondrial protein involved in the biogenesis of iron-sulfur clusters critical to mitochondrial respiratory chain activity.

A number of small molecules, such as histone deacetylase inhibitors and nicotinamide, and large molecules, such as engineered transcription activator-like effectors, are reported as FXV upregulation approaches for FRDA therapy (Gottesfeld J M et al. “Increasing frataxin gene expression with histone deacetylase inhibitors as a therapeutic approach for Friedreich's ataxia.” J of Neurochemistry 126(Suppl 1):147-154 (2013); Soragni E et al. “Epigenetic therapy for Friedreich ataxia.” Annals of Neurology 76:489-508 (2014); Libri V et al. “Epigenetic and neurological effects and safety of high-dose nicotinamide in patients with Friedreich's ataxia: an exploratory, open-label, dose-escalation study.” Lancet 384:504-513 (2014); and Chapdelaine P et al. “A Potential New Therapeutic Approach for Friedreich Ataxia: Induction of Frataxin Expression With TALE Proteins.” Molecular therapy. Nucleic acids 2:e119 (2013)). However, no clear results supporting the benefit of any of these drugs have so far been obtained in randomized controlled trials. Previously, interferon γ-1b treatment option for FRDA to increase FXN levels at a phase 3 randomized controlled trial was dropped following the failure to show any improvement in the outcome measures (ClinicalTrials.gov Identifier: NCT02415127).

Therapeutic development for FRDA currently focuses on (a) symptomatic treatment approaches and (b) finding ways to increase FXN expression. Symptomatic treatment targeting antioxidant defense mechanism via NRF2 activation (omaveloxolone) improved neurological function in FRDA patients with moderate dysfunction (Lynch D R et al. “Safety and Efficacy of Omaveloxolone in Friedreich Ataxia (MOXle Study).” Annals of Neurology 89:212-225 (2021)). However, symptomatic treatment approaches which reverse the cellular sensitivity due to FXN deficiency are not thought to be curative, as the primary FXN deficit will remain.

What is needed is a therapeutic that will increase FXN expression and reverse or lessen symptomatic pathways associated with FRDA.

### SUMMARY

Described are compositions comprising two or more of quercetin, taurine, epigallocatechin gallate (EGCG), and ferrous sulfate. The compositions can be used to treat Friedreich's ataxia (FRDA). Pharmaceutical formulations and methods of using the compositions and pharmaceutical formulations are also described. The compositions, pharmaceutical formulations, and methods can be used to treat subjects suffering from FRDA or to prevent or alleviate one or more symptoms associated with FRDA. The two or more of quercetin, taurine, epigallocatechin gallate (EGCG), and ferrous sulfate can be formulated together and administered to a subject is a single dosage form or the two or more of quercetin, taurine, epigallocatechin gallate (EGCG), and ferrous sulfate can be formulated separately and administered to a subject in two or more separate dosage forms.

In some embodiments, the compositions for treating FRDA contain three or more of: quercetin, taurine, EGCG, and ferrous sulfate comprise. In some embodiments, the compositions for treating FRDA contain: (a) quercetin, taurine, and EGCG; (b) quercetin, taurine, and ferrous sulfate; (c) quercetin, EGCG, and ferrous sulfate; (d) taurine, EGCG, and ferrous sulfate; or (e) quercetin, taurine, EGCG, and ferrous sulfate. In some embodiments, the compositions for treating FRDA contain quercetin, taurine, and EGCG. In some embodiments, the compositions for treating FRDA contain quercetin, taurine, EGCG, and ferrous sulfate. In some embodiments, the compositions for treating FRDA further comprise a pharmaceutically acceptable excipient.

The described compositions for treating FRDA can be formulated for enteral administration, oral administration, parenteral administration, intravascular administration, intravenous administration, rectal administration, intraperitoneal injection, subcutaneous injection, transcutaneous administration, or intramuscular injection. The described compositions for treating FRDA can be formulated as a liquid, an aqueous solution, a suspension, a solid, a powder, a granule, or a pill. Pills include, but are not limited to lozenges, capsules, tablets, and caplets. The described compositions can be formulated for bolus administration or for repeat dosing. Repeat dosing includes, but is not limited to, daily and multiple times per day. Multiple times per day includes, but is not limited to, 2, 3, 4, 5, 6, or more times per day.

The described compositions for treating FRDA can be administered daily, once daily, twice daily, thrice daily, four times per day, once every 2, 3, 4, 5, or 6 days, weekly, biweekly, every four weeks, or monthly.

Also described are methods of treating FRDA comprising administering to a subject having FRDA, diagnosed with FRDA, or at risk of developing FRDA a composition comprising two or more of quercetin, taurine, epigallocatechin gallate (EGCG), and ferrous sulfate. In some embodiments, the composition contains three or more of: quercetin, taurine, EGCG, and ferrous sulfate. In some embodiments, the composition for treating FRDA contains: (a) quercetin, taurine, and EGCG; (b) quercetin, taurine, and ferrous sulfate; (c) quercetin, EGCG, and ferrous sulfate; (d) taurine, EGCG, and ferrous sulfate; or (e) quercetin, taurine, EGCG, and ferrous sulfate. In some embodiments, the composition contains quercetin, taurine, and EGCG. In some embodiments, the composition contains quercetin, taurine, EGCG, and ferrous sulfate. In some embodiments, the compositions for treating FRDA further comprise a pharmaceutically acceptable excipient. In some embodiments, the compositions for treating FRDA further comprise one or more additionally FRDA therapeutics.

The described compositions can be administered to a subject by enteral administration, oral administration, parenteral administration, intravascular administration, intravenous administration, rectal administration, intraperitoneal injection, subcutaneous injection, transcutaneous administration, or intramuscular injection. The described compositions for treating FRDA can be administered to a subject as a liquid, an aqueous solution, a suspension, a solid, a powder, a granule, or a pill. Pills include, but are not limited to lozenges, capsules, tablets, and caplets. The described compositions can be administered to a subject as bolus administration or using repeat dosing. Repeat dosing includes, but is not limited to, daily and multiple times per day. Multiple times per day includes, but is not limited to, 2, 3, 4, 5, 6, or more times per day. The described compositions for treating FRDA can be administered to a subject daily, once daily, twice daily, thrice daily, four times per day, once every 2, 3, 4, 5, or 6 days, weekly, biweekly, every four weeks, or monthly.

In some embodiments, the subject is homozygous for a GAA trinucleotide repeat expansion within the first intron of the FXN gene. In some embodiments, the subject has a mutation in the FXN gene other than a the GAA trinucleotide repeat expansion. In some embodiments, the subject contains a GAA trinucleotide repeat expansion within the first intron of one copy of their FXN gene and another mutation in the other copy of their FA gene. In some embodiments, the subject contains mutations other than GAA trinucleotide repeat expansions within the first intron in both copies of their FXN genes.

In some embodiments, a described composition for treating FRDA is administered to a subject prior to appearance of one or more symptoms associated with FRDA. In some embodiments, a described composition for treating FRDA is administered to a subject subsequent to appearance of one or more symptoms associated with FRDA. Administration of a composition for treating FRDA to a subject can prevent or delay onset of the one or more symptoms associated with FRDA. Administration of a composition for treating FRDA to a subject can reduce the severity or delay progression of the one or more symptoms associated with FRDA. Symptoms associated with FRDA include, but are not limited to: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block. In some embodiments, the described compositions for treating FRDA can increase survival time of the subject.

Also described are methods of modulating expression of one or more genes dysregulated in FRDA comprising administering to a subject having FRDA, diagnosed with FRDA, or at risk of developing FRDA a composition comprising two or more of quercetin, taurine, epigallocatechin gallate (EGCG), and ferrous sulfate. In some embodiments, the composition contains three or more of: quercetin, taurine, EGCG, and ferrous sulfate. In some embodiments, the composition for treating FRDA contains: (a) quercetin, taurine, and EGCG; (b) quercetin, taurine, and ferrous sulfate; (c) quercetin, EGCG, and ferrous sulfate; (d) taurine, EGCG, and ferrous sulfate; or (e) quercetin, taurine, EGCG, and ferrous sulfate. In some embodiments, the composition contains quercetin, taurine, and EGCG. In some embodiments, the composition contains quercetin, taurine, EGCG, and ferrous sulfate. In some embodiments, the compositions for treating FRDA further comprise a pharmaceutically acceptable excipient. Genes dysregulated in FRDA include, but are not limited to, ACO1, AKT1, ALAS2, ATF4, AVEN, BIRC5, CASP8, CAT, CCL6, CFL1, CXCL1, CYGB, EGR1, FXN, GDF15, HFE, HMOX1, IFITM3, IGF1, IREB2, MMP2, MYD88, NRF2, PDHA1, RELA, SLC25A28, SLC40A1, SOD1, STAT3, TFB1M, TFRC, TGFBR2, TNFRSF1A, TRP53, TUG1, and VCAM1. Modulating expression of the one or more gene dysregulated in FRDA can be used to treat or prevent one or more symptoms associated with FRDA.

In some embodiments, the described compositions increase expression of the fxn gene. In some embodiments, the described compositions increase expression of the fxn gene by about 2 to about 10 fold or more. In some embodiments, the described compositions increase expression of the FXN protein. In some embodiments, the described compositions increase fxn expression such that the FXN protein is present at about 50% or more of the FXN protein level present in a control asymptomatic heterozygous (FXN/fxn) subject.

In some embodiments, the described compositions increase expression of the fxn gene in a subject suffering from, diagnosed with, or at risk of developing FRDA. In some embodiments, the described compositions increase expression of the fxn gene by about 2 to about 10 fold or more in a subject suffering from, diagnosed with, or at risk of developing FRDA. In some embodiments, the described compositions increase expression of the FXN protein in a subject suffering from, diagnosed with, or at risk of developing FRDA. In some embodiments, the described compositions increase fxn expression such that the FXN protein is present in a subject suffering from, diagnosed with, or at risk of developing FRDA at about 50% or more of the FXN protein level present in a control asymptomatic heterozygous (FXN/fxn) subject.

In some embodiments, the described compositions increase expression of the fxn gene in fibroblast cells. In some embodiments, the described compositions increase expression of the fxn gene by about 2 to about 10 fold or more in fibroblast cells. In some embodiments, the described compositions increase expression of the FXN protein in fibroblast cells.

In some embodiments, the described compositions increase expression of the fxn gene in fibroblast cells from a subject suffering from, diagnosed with, or at risk of developing FRDA. In some embodiments, the described compositions increase expression of the fxn gene by about 2 to about 10 fold or more in fibroblast cells from a subject suffering from, diagnosed with, or at risk of developing FRDA. In some embodiments, the described compositions increase expression of the FXN protein in fibroblast cells from a subject suffering from, diagnosed with, or at risk of developing FRDA.

In some embodiments, the described compositions improve cardiac function in a subject suffering from, diagnosed with, or at risk of developing FRDA.

In some embodiments, the described compositions increase survival in a subject suffering from, diagnosed with, or at risk of developing FRDA

The described combinations and methods target iron metabolism, mitochondrial dysfunction, and immune pathways. In addition to FRDA, other conditions are known to involve iron metabolism pathways, mitochondrial dysfunction pathways, and immune pathways. Such diseases can also be treated with the disclosed compositions. The diseases that can be treated with the described compositions include, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, and spinocerebellar ataxia.

Also described are kits containing the described compositions for treating FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, and spinocerebellar ataxia.

### BRIEF DESCRIPTION OF THE DRAWINGS

FIG. 1A-C. Stressor pathways are dysregulated due to FXN knockdown in FRDAkd mice. Networks highlighting top differentially expressed genes due to FXN knockdown in FRDAkd mice associated with the stressor pathways. Nodes=genes, edges=correlation >0.5, red=upregulation, green=downregulation.

FIG. 1D. Stressor pathways are dysregulated due to FXNknockdown in FRDAkd mice and role of quercetin, taurine, and EGCG in treating the different dysregulated pathways.

FIG. 2A. Graphs illustrating expression levels of frataxin (FXN) and various iron metabolism-related genes in control fibroblasts (age and sex matched) or fibroblasts from a patient with Friedreich's Ataxia (FRDA) treated with vehicle or 75 μM quercetin. Expression levels were determined using Real-time PCR and normalized to Hprtl. Experiments were conducted in triplicates and values expressed as mean±SEM. One-way ANOVA and 0.05**

FIG. 2B. Graphs illustrating dose-dependent effects of quercetin (Q) treatment on the FRDA-associated marker genes FXN and NRF2, as determined by qPCR in fibroblasts from patients with FRDA. N=3. One-way ANOVA and Welch's t-test, ±SEM. *p≤0.05, **p≤0.01, ***p 0.001.

FIG. 3A-B. Western blot analysis of FXN and NRF2 in FRDA cells and FRDAkd mice after quercetin treatment. (A) Increased expression of FXN and NRF2 after 24 hours quercetin treatment in FRDA fibroblast cell line at two different doses (75 μM and 100 μM). (B) Increased expression of FXN and NRF2 after three weeks of quercetin treatment in FRDAkd mice liver. Three FRDAkd mice without doxycycline (Tg No Dox, control) and three mice treated with 625 mg/kg doxycycline (Tg Low Dox (FXN knockdown)+Quercetin) plus 35 mg/kg/day of quercetin showed increased FXN and NRF2 levels.

FIG. 3C-D. Western blot analysis of FXN and NRF2 in FRDA cells and FRDAkd mice after quercetin treatment. (C) Increased expression of FXN and NRF2 after eight weeks of quercetin treatment in FRDAkd mice heart. Tg Low Dox animals were treated with 625 mg/kg doxycycline (FXN knockdown) and Tg Low Dox+Quercetin animal was provided with 625 mg/kg doxycycline plus 35 mg/kg/day of quercetin. (D) Independent replication showing increased expression of FXN after 24 hours quercetin treatment in FRDA fibroblast cell line at 7 μM dose. Overall, quercetin increased the expression of FXN and NRF2 in both in vitro and in vivo conditions.

FIG. 3E-F. Graphical representation of the increases in expression of FXN and NRF2 in FRDAkd mouse heart (G) and FRDA cells (H) after quercetin (Q) treatment shown in FIG. 3A-B.

FIG. 3G-H. Graphical representation of the increases expression of FXN in FRDA cells (G) and FRDAkd mice (F) after quercetin (Q) treatment shown in FIG. 3C-D.

FIG. 4. Plot showing increase survival in FRDA animal treated with quercetin, taurine, and EGCG. FRDAkd mice (DOX) showed significantly reduced survival at 17 weeks. No mortality was observed FRDAkd mice (DOX) treated with quercetin, taurine, and EGCG. FRDAkd mice were treated with doxycycline (DOX for FXN knockdown) or with doxycycline plus 100 mg/kg/day taurine, 35 mg/kg/day quercetin, and 4.6 mg/kg/day EGCG, and 0.5 mg/kg/day of ferrous sulfate for 16 weeks. N=10 animals per group.

FIG. 5. Effects of combinatorial drug treatment (quercetin, taurine, EGCG, and ferrous sulfate) on the FA-genes. Relative mRNA expression of the FA-genes in the heart (top) and spinal cord (bottom) from FRDAkd mice treated with no doxycycline (Tg NO Dox), or doxycycline (Tg+Dox for FXN knockdown) and with doxycycline plus 100 mg/kg/day taurine, 35 mg/kg/day quercetin, and 4.6 mg/kg/day EGCG, and 0.5 mg/kg/day of ferrous sulfate (Tg+TQE+Fe+Dox) for 16 weeks. N=3 animals per group. Only the FA genes that showed significant changes are shown. Two-way ANOVA was performed. Data are shown mean±SEM. Comparable results were observed in the absence of ferrous sulfate. In order for each group (gene): Tg NO Dox, TG+Dox, and Tg+TQE+Fe+Dox.

FIG. 6. Graphs illustrating AKT1, ALAS2, and ATF4 gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 7. Graphs illustrating CASP8, CAT, and CXCL1 gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 8. Graphs illustrating CYGB, FXN, and HFE gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 9. Graphs illustrating HMOX1, IGF1, and MYD88 gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 10. Graphs illustrating NRF2, RELA, and SLC40A1 gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 11. Graphs illustrating SOD1, STAT3, and TFRC gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 12. Graphs illustrating TRP53, TUG1, and VCAM1 gene expression in various tissues in the transgenic FRDAkd mice without doxycycline (Tg NO Dox), FRDAkd mice with doxycycline for FXN knockdown (Tg+Dox), and FRDAkd mice treated with doxycycline plus quercetin, taurine, EGCG, and ferrous sulfate (Tg+TQE+Fe+Dox). N=3 animals per group. Two-way ANOVA was performed to determine the significance. Data are shown as Mean±SD (*p≤0.05). In order for each group (tissue): Tg NO Dox, Tg+Dox, Tg+TQE+Fe+Dox.

FIG. 13. Graphs illustrating the effects of QTE treatment on FRDA-associated marker genes. Relative mRNA expression of marker genes in hearts from FRDAkd mice treated with no dox (first bar in each group), dox (−Fxn KD, middle bar in each group), or dox plus QTE (third bar in each group) for 6 weeks with (doses in mg/kg/day: Q=35, T=100, E=4.6). N=4 animals per group. Two-way ANOVA was performed, ±SEM. In order for each group (gene): FRDAkd No Dox, FRDAkd Dox (FXN KD)—no drug treatment, FRDAkd Dox (FXN KD)+QTE therapy.

FIG. 14A. ECG graphs illustrating cardiac deficits induced by FXN KD in FRDAkd mice. ECG recordings from animals after 24 weeks of dox treatment, showing long QT intervals in Tg-Dox mice. All results are statistically significant.

FIG. 14B. Graph illustrating QT and corrected QT intervals in untreated Tg-No-Dox and Tg-Dox animals and Tg-Dox animals receiving QTE therapy for 16 weeks. N=8-10 animals per group. Values represent the mean±SME. One-way ANOVA test *=P<0.05

### DETAILED DESCRIPTION

### I. Definitions

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

“Subject” refers to an animal, such as a mammal, for example a human. The methods described herein can be useful in both humans and non-human animals. In some embodiments, the subject is a mammal (such as an animal model of disease). Mammal includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treat,” “treatment,” and the like, mean the methods or steps taken to provide relief from or alleviation of the number, severity, and/or frequency of one or more symptoms of a disease or condition in a subject. The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy.

An “effective amount” refers to an amount that is capable of treating or ameliorating a disease or condition or otherwise capable of producing an intended therapeutic effect.

A “pharmacologically effective amount,” “therapeutically effective amount,” “effective amount,” or “effective dose” refers to that amount of an agent to produce the intended pharmacological, therapeutic, or preventive result, such as, but not limited to, treating or ameliorating a disease or condition.

### II. Compositions for Treating Friedreich's Ataxia

Described are compositions for use in the treatment of Friedreich's ataxia (FRDA). The compositions can be used to treat a subject having FRDA, diagnosed with FRDA, at risk of developing FRDA, or genetically predisposed to FRDA. FRDA is caused by severely reduced levels of frataxin (FXN). The reduced levels of FXN can be due to a GAA trinucleotide repeat expansion within the first intron of the FXN gene or other mutations in the FXN gene. Patients having mutations in both copies of the FXN gene, such as subjects homozygous for the GAA trinucleotide repeat expansion within the first intron of the FXN gene, are at risk of developing FRDA or predisposed to developing FRDA. While patients diagnosed with FRDA are often homozygous for a GAA trinucleotide repeat expansion within the first intron of the FXN gene, FRDA patients with FRDA or at risk of developing FRDA can also have other mutations or defects in one or both copies of their FXN genes.

The described compositions for treating FRDA comprise two or more of quercetin, taurine, epigallocatechin gallate (EGCG), and ferrous sulfate. In some embodiments, compositions for treating FRDA comprise three or more of quercetin, taurine, EGCG, and ferrous sulfate. In some embodiments, compositions for treating FRDA comprise quercetin, taurine, and EGCG. In some embodiments, compositions for treating FRDA comprise quercetin, taurine, EGCG, and ferrous sulfate. Pharmaceutical compositions and methods of using the compositions and pharmaceutical compositions are also described. The compositions, pharmaceutical compositions, and methods can be used to treat subjects suffering from FRDA or to prevent or alleviate one or more symptoms associated with FRDA.

Quercetin (3,3′,4′,5,7-Pentahydroxyflavone; CAS #117-39-5) is a plant flavonol, found in fruits, vegetables, and grains.

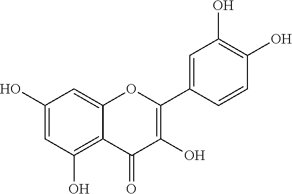 [SMILES: O=c1c(O)c(-c2ccc(O)c(O)c2)oc2cc(O)cc(O)c12]

Taurine (2-Aminoethane-1-sulfonic acid; CAS #107-35-7) is a semi-essential amino acid naturally occurring in animal tissue and normally present in bile.

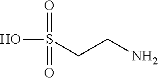

Epigallocatechin gallate (EGCG, (2R,3R)-3′,4′,5,5′,7-Pentahydroxyflavan-3-yl 3,4,5-trihydroxybenzoate, CAS #989-51-5) is a polyphenol found in green tea.

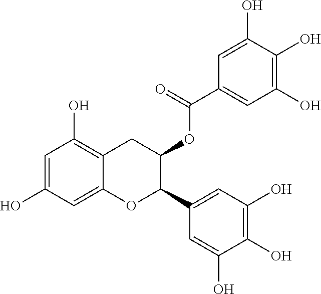 [SMILES: O=C(O[C@@H]1Cc2c(O)cc(O)cc2O[C@@H]1c1cc(O)c(O)c(O)c1)c1cc(O)c(O)c(O)c1]

Quercetin, taurine, and EGCG are found in dietary supplements and have generally recognized as safe (GRAS) status from the FDA, indicating relatively low toxic potential.

Ferrous sulfate (iron(II) sulfate, FeSO₄·xH₂O; e.g., FeSO₄·7H₂O; CAS #7782-63-0) is a hydrate of iron sulfate and is commonly prescribed as an iron supplement to treat iron deficiency and iron-deficiency anemia.

Combining these compounds was found to provide synergistic effects in treating FRDA and symptoms associated with FRDA. A combination of quercetin, taurine, and EGCG was found increase survival in mouse FRDA models. Treatment of animals individually with quercetin, taurine, or EGCG did not improve survival rates.

The described combinations and methods target iron metabolism, mitochondrial dysfunction, and immune pathways. In addition to FRDA, other conditions are known to involve iron metabolism pathways, mitochondrial dysfunction pathways, and immune pathways. Such diseases can also be treated with the disclosed compositions. The diseases that can be treated with the described compositions include, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, and spinocerebellar ataxia.

In addition to the combination of quercetin, taurine, and epigallocatechin gallate (EGCG), other combinations that similarly affect iron metabolism, mitochondrial dysfunction, and immune pathways may also be used to treat to FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia.

Also described are methods for treating a subject suffering from or at risk of developing FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the method comprising administering to the subject a one or more first compounds that regulate iron metabolism, one or more second compounds that modulate immune function, and one or more third compounds that reduce mitochondrial dysfunction. The first compound can be, but is not limited to, quercetin or ferrous sulfate. The second compound can be, but is not limited to, epigallocatechin gallate. The third compound can be, but is not limited to, taurine.

### III. Formulations

The described combinations can be administered either alone or in combination with pharmaceutically acceptable carriers, excipients, or diluents, in a pharmaceutical composition. The composition may be a pharmaceutical composition suitable for in vivo delivery to a subject. IN some embodiments, the quercetin, taurine, epigallocatechin gallate (EGCG) are formulated together and administered to a subject is a single dosage form. In some embodiments, the quercetin, taurine, epigallocatechin gallate (EGCG) are formulated separately and administered to a subject in two or three separate dosage forms.

A pharmaceutical composition includes a pharmacologically effective amount of two or more of quercetin, taurine, and epigallocatechin gallate as active ingredients, and optionally one or more pharmaceutically acceptable excipients, and optionally one or more additional therapeutic agents. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, bulking agents, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical composition comprises a single dosage form containing two or more of quercetin, taurine, EGCG, and ferrous sulfate. In some embodiments, the pharmaceutical composition comprises a single dosage form containing three or more of quercetin, taurine, EGCG, and ferrous sulfate. In some embodiments, the pharmaceutical composition comprises a single dosage form containing quercetin, taurine, and EGCG. In some embodiments, the pharmaceutical composition comprises a single dosage form containing quercetin, taurine, EGCG, and ferrous sulfate.

In some embodiments, the pharmaceutical composition comprises a dosage form suitable for administering to a subject quercetin at about 35 mg/kg/day, taurine at about 100 mg/kg/day, and EGCG at about 4.6 mg/kg/day. The dosage form can further contain ferrous sulfate in an amount suitable for administering to the subject the ferrous sulfate at about 0.5 mg/kg/day. The dosage for can be formulated to deliver the quercetin, taurine and EGCG, and optionally ferrous sulfate, as a single dose or in multiple doses.

For treatment of mouse, a pharmaceutically effective amount of taurine is 100 mg/kg/day, a pharmaceutically effective amount of quercetin 35 mg/kg/day, a pharmaceutically effective amount of epigallocatechin gallate is 4.6 mg/kg/day, and a pharmaceutically effective amount of ferrous sulfate is 0.5 mg/kg/day. The treatment of other mammals, such a human, the dosage is adjusted accordingly.

In some embodiments, a human equivalent dose (HED), is calculated using the formula:

Human Equivalent Dose (HED) (mg/kg)=Animal dose (mg/kg)×Km ratio (0.081).

Using the above formula, a dose of 100 mg/kg/day taurine in mouse corresponds to an HED of 100 mg/kg/day×0.081=8.1 mg/kg/day, or 486 mg/d for a 60 kg human. Using the above formula, a dose of 35 mg/kg/day quercetin in mouse corresponds to an HED of 35 mg/kg/day×0.081=2.835 mg/kg/day, or 170 mg/d for a 60 kg human. Using the above formula, a dose of 4.6 mg/kg/day EGCG in mouse corresponds to an HED of 4.6 mg/kg/day×0.081=0.3726 mg/kg/day, or 22 mg/d for a 60 kg human. Using the above formula, a dose of 0.5 mg/kg/day ferrous sulfate in mouse corresponds to an HED of 0.5 mg/kg/day×0.081=0.0405 mg/kg/day, or 2.43 mg/d for a 60 kg human. An effective pharmaceutical dose for each drug can be determined empirically and optimized using methods standard in the art.

In some embodiments, the pharmaceutical composition comprises a dosage form suitable for administering to a subject quercetin at about 2450 mg/day, taurine at about 7000 mg/day, and EGCG at about 322 mg/day. The dosage form can further contain ferrous sulfate in an amount suitable for administering to the subject the ferrous sulfate at about 35 mg/kg/day. The dosage for can be formulated to deliver the quercetin, taurine and EGCG, and optionally ferrous sulfate, as a single dose or in multiple doses.

In some embodiments, the pharmaceutical composition comprises a dosage form suitable for administering to a subject quercetin at about 1500 to about 2500 mg/day, taurine at about 4000 to about 7000 mg/day, and EGCG at about 200 to about 350 mg/day. The dosage form can further contain ferrous sulfate in an amount suitable for administering to the subject the ferrous sulfate at about 20-35 mg/day. The dosage for can be formulated to deliver the quercetin, taurine and EGCG, and optionally ferrous sulfate, as a single dose or in multiple doses.

In some embodiments, the pharmaceutical composition comprises a dosage form containing dosage levels adjusted appropriately for administration to children.

In some embodiments, the pharmaceutical composition further comprises one or more additional therapeutics. In some embodiments, the pharmaceutical composition further comprises one or more additional FRDA therapeutics.

The described pharmaceutical compositions can be formulated as liquid formulations or as solid formulations (including powders or lyophilized formulations). The described pharmaceutical compositions can be provided as, for example, a powder, granule, liquid, aqueous solution, suspension, cream, ointments, or pill. Pills include, but are not limited to, lozenges, capsules, tablets, and caplets. A pharmaceutical composition suitable for oral administration can be provided as a powder, granule, liquid, suspension, or pill. The described pharmaceutical compositions may be provided in extending release formulations or a bolus formulations. The described pharmaceutical compositions may be provided for continuous infusion over a period of minutes to hours.

The described pharmaceutical compositions can be formulated for repeat dosing. The described pharmaceutical compositions can be formulated for administration once per month, once every two weeks, once be week, once per day, twice per pay, three times per day, four times per day, or every 3, 4, 6, 8, or 12, or 24 hours. The described pharmaceutical compositions may be provided in unit-dose or multi-dose containers or pills.

### IV. Administration

The pharmaceutical compositions can be administered to a subject via enteral, parenteral, inhalational, transdermal, transmucosal, sublingual, buccal, and topical routes. Enteral administration includes, but is not limited to, oral, gastric or duodenal feeding tube, rectal suppository, and rectal enema. Parenteral administration includes, but is not limited to, injection, infusion, intraarterial, intracardiac, intradermal, intraduodenal, intramedullary, intramuscular, intraosseous, intraperitoneal, intrathecal, intravascular, intravenous, intravitreal, epidural, and subcutaneous administration. Topical administration includes, but is not limited to, epicutaneous, dermal, enema, eye drops, ear drops, intranasal, vaginal administration.

The described pharmaceutical compositions are administered in an amount effective for treatment, amelioration, or prevention of one or more symptoms or conditions associated with FRDA. An effective amount can be an amount that is capable of at least partially preventing or reversing at least one symptom or condition associated with FRDA. Treating, ameliorating, or preventing includes slowing progression of FRDA or stabilizing a symptom or condition such that is does not get worse. The dose required to obtain an effective amount may vary depending on the agent, formulation, disease or disorder, and individual to whom the agent is administered. Determination of an effective amounts may involve an in vitro assay in which varying doses of agent are administered to cells in culture and the concentration of agent effective for ameliorating some or all symptoms is determined in order to calculate the concentration required in vivo. Determination of an effective amounts may be based on in vivo animal studies.

The described pharmaceutical compositions are administered in an amount effective for treatment, amelioration, or prevention of one or more symptoms or conditions associated with Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia.

In some embodiments, an effective dose of quercetin is about 50 to about 3000 mg, about 100 to about 3000 mg, about 200 to about 3000 mg, about 250 to about 3000 mg, about 300 to about 3000 mg, about 400 to about 3000 mg, about 500 to about 3000 mg, or about 1000 to about 3000 mg. In some embodiments, an effective dose of quercetin is about 50 to about 3000 mg, about 100 to about 3000 mg, about 200 to about 3000 mg, about 250 to about 3000 mg, about 300 to about 3000 mg, about 400 to about 3000 mg, about 500 to about 3000 mg, or about 1000 to about 3000 mg per day. In some embodiments, an effective dose of quercetin is about 2400 to about 2500 mg per day. Larger doses may be appropriate if not given every day. In some embodiments, an effective dose of quercetin is about 20 to about 45 mg/kg/day. In some embodiments, an effective dose of quercetin is about 25 to about 40 mg/kg/day. In some embodiments, an effective dose of quercetin is about 25, about 26, about 27, about 28, about 29, about 30, about 31, about 32, about 33, about 34, about 35, about 36, about 37, about 38, about 39, or about 40 mg/kg/day. In some embodiments, an effective dose of quercetin is about 35 mg/kg/day.

In some embodiments, an effective dose of EGCG is about 50 to about 1000 mg, about 100 to about 1000 mg, about 200 to about 1000 mg, about 250 to about 1000 mg, about 300 to about 1000 mg, about 400 to about 1000 mg, or about 500 to about 1000 mg. In some embodiments, an effective dose of EGCG is about 50 to about 1000 mg, about 100 to about 1000 mg, about 200 to about 1000 mg, about 250 to about 1000 mg, about 300 to about 1000 mg, about 400 to about 1000 mg, or about 500 to about 1000 mg per day. In some embodiments, an effective dose of EGCG is about 300 to about 750 mg/day. In some embodiments, an effective dose of EGCG is about 700 mg/day. In some embodiments, an effective dose of EGCG is about 300-350 mg/day. Larger doses may be appropriate if not given every day. In some embodiments, an effective dose of EGCG is about 3 to about 10 mg/kg/day. In some embodiments, an effective dose of EGCG is about 3, about 4, about 5, about 6, about 7, about 8, about 9, or about 10 mg/kg/day. In some embodiments, an effective dose of EGCG is about 4 to about 6 mg/kg/day. In some embodiments, an effective dose of EGCG is about 4, about 4.1, about 4.2, about 4.3, about 4.4, about 6 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/kg/day. In some embodiments, an effective dose of EGCG is about 4.6 mg/kg/day

In some embodiments, an effective dose of taurine is about 100 to about 7000 mg, about 200 to about 7000 mg, about 250 to about 7000 mg, about 300 to about 7000 mg, about 400 to about 7000 mg, about 500 to about 7000 mg, or about 1000 to about 7000 mg. In some embodiments, an effective dose of taurine is about 100 to about 7000 mg, about 200 to about 7000 mg, about 250 to about 7000 mg, about 300 to about 7000 mg, about 400 to about 7000 mg, about 500 to about 7000 mg, or about 1000 to about 7000 mg per day. Larger doses may be appropriate if not given every day. In some embodiments, an effective dose of taurine is about 50 to about 150 mg/kg/day. In some embodiments, an effective dose of taurine is about 50, about 60, about 70, about 80, about 90, about 100, about 110, about 120, about 130, about 140, about 150 mg/kg/day. In some embodiments, an effective dose of taurine is about 80 to about 120 mg/kg/day. In some embodiments, an effective dose of taurine is about 100 mg/kg/day.

In some embodiments, an effective dose of ferrous sulfate is about 10 mg to about 1000 mg, about 10 mg to about 500 mg, about 10 mg to about 400 mg, about 10 mg to about 300 mg, about 10 mg to about 200 mg, about 10 mg to about 100 mg, about 10 to about 50 mg, about 20 to about 50 mg, or about 30 to about 50 mg. In some embodiments, an effective dose of ferrous sulfate is about 10 to about 50 mg, about 20 to about 50 mg, or about 30 to about 50 mg per day. In some embodiments, an effective dose of ferrous sulfate is about 0.1 to about 10 mg/kg/day. In some embodiments, an effective dose of ferrous sulfate is about 0.1, about 0.2, about 0.3, about 0.4, about 0.5, about 0.6, about 0.7, about 0.8, about 0.9, or about 1 mg/kg/day. In some embodiments, an effective dose of ferrous sulfate is about 0.5 mg/kg/day.

For combinations of two or more of quercetin, taurine, EGCG, and ferrous sulfate, the above dosage levels of the individual compounds can be combined. For combinations of three or more of quercetin, taurine, EGCG, and ferrous sulfate, the above dosage levels of the individual compounds can be combined. For combinations of quercetin, taurine, and EGCG, the above dosage levels of the individual compounds can be combined. For combinations of quercetin, taurine, EGCG, and ferrous sulfate, the above dosage levels of the individual compounds can be combined. In some embodiments, a subject is administered quercetin at about 35 mg/kg/day, taurine at about 100 mg/kg/day, and EGCG at about 4.6 mg/kg/day. In some embodiments, a subject is administered quercetin at about 2450 mg/day, taurine at about 7000 mg/day, and EGCG at about 322 mg/day. In some embodiments, a subject is administered quercetin at about 1500 to about 2500 mg/day, taurine at about 4000 to about 7000 mg/day, and EGCG at about 200 to about 350 mg/kg/day. In some embodiments, a subject may be additionally administered ferrous sulfate at about 0.5 mg/kg/day. In some embodiments, a subject may be additionally administered ferrous sulfate at about 35 mg/day. In some embodiments, a subject may be additionally administered ferrous sulfate at about 20 to about 35 mg/day. These dosage levels may be adjusted appropriately for administration to children.

### V. Kits

In some embodiments, kits containing the described pharmaceutical compositions are described. The kits comprises two or more of quercetin, taurine, and EGCG, and instructions for administering and/or treating FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia. In some embodiments, the kit comprises quercetin, taurine, and epigallocatechin gallate and instructions for administering and/or treating FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia.

Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications and/or warnings concerning the use the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

In some embodiments, a kit comprises two or more components, including at least one described pharmaceutical composition and instructions for preparation of the dosage form by the patient or person administering the pharmaceutical compositions to the patient. In some embodiments, a kit may further comprise optional components that aid in the administration of the unit dose to a subject, including but not limited to: vials for reconstituting powder forms, syringes for injection, customized IV delivery systems, inhalers, etc. Additionally, a kit can contain instructions for preparation and administration of the compositions. The kit can be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

### VI. Therapeutic Use

The described pharmaceutical compositions can be administered prior to or subsequent to the appearance of one or more symptoms associated with FRDA. In some embodiments, a described pharmaceutical composition is administered to a subject that has received a positive test for being susceptible to or predisposed to development of FRDA. The test can be, but is not limited to, a genetic test. In some embodiments, a described pharmaceutical composition is administered to a subject who has a genotype which predisposes the subject to FRDA. In some embodiments, a described pharmaceutical composition is administered to a subject that has been diagnosed with FRDA.

The described pharmaceutical compositions can be administered prior to or subsequent to the appearance of one or more symptoms associated with Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia. In some embodiments, a described pharmaceutical composition is administered to a subject that has received a positive test for being susceptible to or predisposed to development of Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia. In some embodiments, a described pharmaceutical composition is administered to a subject that has been diagnosed with Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia.

Described are methods of treating, reducing, or preventing, or delay the onset of one or more symptoms associated with Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the method comprising administered to the subject a pharmaceutical composition comprising quercetin, taurine, and epigallocatechin gallate.

Described are methods of treating FRDA, treating or preventing one or more symptoms associated with FRDA, or reducing the severity of one or more symptoms associated with FRDA, the method comprising administered to the subject a pharmaceutical composition comprising quercetin, taurine, and epigallocatechin gallate.

In some embodiments, the described pharmaceutical compositions can be administered to a subject to ameliorate one or more symptoms associated with FRDA in a subject diagnosed with FRDA. The symptoms can be, but are not limited to: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.

In some embodiments, the described pharmaceutical compositions can be administered to a subject to reduce one or more symptoms associated with FRDA in a subject diagnosed with FRDA. The symptoms can be, but are not limited to: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.

In some embodiments, the described pharmaceutical compositions can be administered to a subject to prevent or reduce the risk of developing or delay the onset or progression of one or more symptoms associated with FRDA in a subject diagnosed with FRDA or at risk of developing FRDA. The symptoms can be, but are not limited to: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.

In some embodiments, a subject is at risk of developing FRDA if the subject contains defects in both copies of the FXN gene. The defects in the subjects FXN genes can be a GAA trinucleotide repeat expansion within the first intron of the FXN gene, or other loss of function mutations in the FXN gene.

In some embodiments, the described pharmaceutical compositions can be administered to a subject to treat one or more conditions associated with FRDA, prevent one or more conditions cause associated with FRDA, or reduce the severity of one or more conditions associated with FRDA in subject diagnosed with FRDA or a risk of developing FRDA. Treating includes, but is not limited to, reducing severity of the condition, reducing one or more symptoms associated with the condition, or delaying progression of the condition. The conditions can be, but are not limited to: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.

In some embodiments, methods for modulating expression of one or more genes dysregulated in Friedreich's ataxia are described, the methods comprise administering to a subject any of the described pharmaceutical compositions comprising two or more of quercetin, taurine, and EGCG. In some embodiments, the pharmaceutical composition comprises quercetin, taurine, and EGCG. In some embodiments, the pharmaceutical composition further comprises ferrous sulfate. The one or more genes includes, but is not limited to, ACO1, AKT1, ALAS2, ATF4, AVEN, BIRC5, CASP8, CAT, CCL6, CFL1, CXCL1, CYGB, EGR1, FXN, GDF15, HFE, HMOX1, IFITM3, IGF1, IREB2, MMP2, MYD88, NRF2, PDHA1, RELA, SLC25A28, SLC40A1, SOD1, STAT3, TFB1M, TFRC, TGFBR2, TNFRSF1A, TRP53, TUG1, and VCAM1.

Modulating expression can comprise increasing expression of the gene or decreasing expression of the gene. Expression of the gene can be increased or decreased in the subject, in a tissue of the subject, or in a cell type of the subject. Expression of the gene can be increased or decreased in the subject, in a tissue of the subject, or in a cell type in the subject relative to expression of the gene in the subject, in the tissue of the subject, or in the cell type in the subject prior to administration of the pharmaceutical composition. Expression of the gene can be increased or decreased in the subject, in a tissue of the subject, or in a cell type in the subject relative to a predetermined control. The predetermined control can be derived from expression of the gene in FRDA patients, tissue from FRDA patients, or cell types from FRDA patients that have not received the pharmaceutical composition. The tissue can be, but is not limited to, neural tissue, muscle tissue, liver tissue, heart tissue, or spinal tissue. The cell type can be, but is not limited to, neural cells, muscle cells, liver cells, or cardiac cells.

Modulating expression of the one or more gene dysregulated in FRDA can treat or prevent one or more symptoms associated with FRDA.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### VII. Listing of Embodiments

1. A composition for treating Friedreich's ataxia (FRDA) comprising two or more of quercetin, taurine, epigallocatechin gallate, and ferrous sulfate.
2. The composition of embodiment 1, wherein two or more comprises three or more.
3. The composition of embodiment 2, wherein three or more comprises:
   a. quercetin, taurine, and epigallocatechin gallate;
   b. quercetin, taurine, and ferrous sulfate;
   c. quercetin, epigallocatechin gallate, and ferrous sulfate; or
   d. taurine, epigallocatechin gallate, and ferrous sulfate.
4. The composition of embodiment 3 wherein the composition comprises quercetin, taurine, and epigallocatechin gallate.
5. The composition of embodiment 4, wherein the composition further comprises ferrous sulfate.
6. The composition of any one of embodiments 1-5, further comprising a pharmaceutically acceptable excipient.
7. The composition of any one of embodiments 1-6, wherein the composition is for formulated for enteral administration, oral administration, parenteral administration, intravascular administration, intravenous administration, rectal administration, intraperitoneal injection, subcutaneous injection, transcutaneous administration, or intramuscular injection.
8. The composition of any one of embodiments 1-7, wherein the composition is formulated as a liquid, an aqueous solution, a suspension, a solid, a powder, a granule, or a pill.
9. The composition of embodiment 8, wherein the pill comprises a lozenge, a capsule, a tablet, or a caplet.
10. The composition of any one of embodiments 1-9, wherein the composition is formulated for repeat dosing.
11. A method of treating FRDA comprising administering to a subject having FRDA, diagnosed with FRDA, or at risk of developing FRDA the composition of any one of embodiments 1-10.
12. The method of embodiment 11, wherein the subject has loss of function mutations in both copies of their FA genes, where in the loss of function mutations are independently selected from the group comprising: a GAA trinucleotide repeat expansion within the first intron of the FXN gene, a nonsense mutation, a frame shift mutation, insertion, deletion, or missense mutation that reduces function of the encoded frataxin.
13. The method of embodiment 11 or 12, wherein the composition is administered to the subject prior to appearance of one or more symptoms associated with FRDA.
14. The method of embodiment 11 or 12, wherein the composition is administered to the subject subsequent to appearance of one or more symptoms associated with FRDA.
15. The method of embodiment 13, wherein administering the composition to the subject prevents or delays onset of the one or more symptoms associated with FRDA.
16. The method of embodiment 14, wherein administering the composition to the subject reduces the severity or delays progression of the one or more symptoms associated with FRDA.
17. The method of any one of embodiments 13-16, wherein the one or more symptoms associated with FRDA are selected from the group consisting of: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.
18. The method of any one of embodiments 11-17, wherein treating the subject increases survival of the subject.
19. A method for modulating expression of one or more genes dysregulated in Friedreich's ataxia comprising administering to a subject having FRDA, diagnosed with FRDA, or at risk of developing FRDA the composition of any one of embodiments 1-10.
20. The method of embodiment 19, wherein the one or more genes is selected from the group consisting of: ACO1, AKT1, ALAS2, ATF4, AVEN, BIRC5, CASP8, CAT, CCL6, CFL1, CXCL1, CYGB, EGR1, FXN, GDF15, HFE, HMOX1, IFITM3, IGF1, IREB2, MMP2, MYD88, NRF2, PDHA1, RELA, SLC25A28, SLC40A1, SOD1, STAT3, TFB1M, TFRC, TGFBR2, TNFRSF1A, TRP53, TUG1, and VCAM1.
21. A kit for treating FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the kit comprising the composition of any one of embodiments 1-9 and instructions for use.
22. A composition for treating or preventing Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the composition comprising quercetin, taurine, and epigallocatechin gallate.
23. A method for treating a subject suffering from or at risk of developing a disease characterized in involving iron metabolism, mitochondrial dysfunction, and immune pathways, the method comprising administering to the subject a first compound that regulates iron metabolism, a second compound that reduces mitochondrial dysfunction, and a third compound that modulates immune function.
24. The method of embodiment 23, wherein the disease is selected from the group consisting of: FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, and spinocerebellar ataxia.
25. The method of embodiment 23 or 24, wherein the first, second and third compounds comprise: quercetin, taurine, and epigallocatechin gallate.
26. A method of increasing FXN expression in a subject having FRDA, diagnosed with FRDA, or at risk of developing FRDA, comprising administering to the subject the composition of any one of embodiments 1-10.
27. A method of increasing iron metabolism, reducing mitochondrial dysfunction, and modulating immune function in a subject comprising administering to the subject the composition of any one of embodiments 1-10.

### EXAMPLES

### Example 1. Genomic Computational Analyses in FRDA

The protein-coding sequence of FXN is normal in most FRDA patients. FRDA is caused by severely reduced levels of frataxin (FXN) resulting from a GAA trinucleotide repeat expansion within the first intron of the FXN gene. An FRDAkd mouse has been developed that knocks down FXN expression in response to doxycycline (DOX) treatment. Experiments in this FRDAkd mouse model indicate that FXN knockdown leads to various FRDA-associated deficits, including development of clinical and pathological features mimicking those observed in patients with FRDA (Chandran V et al. “Inducible and reversible phenotypes in a novel mouse model of Friedreich's Ataxia.” eLife 6 (2017))

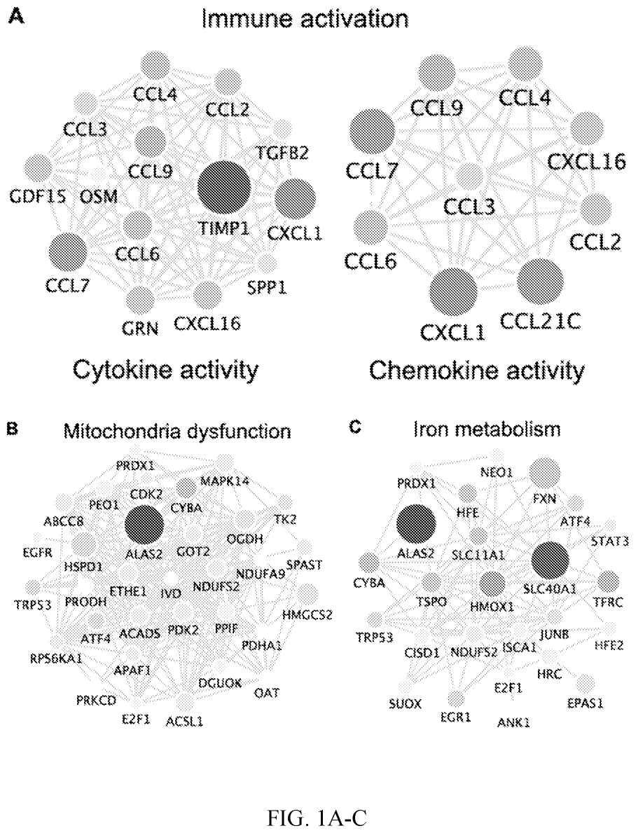

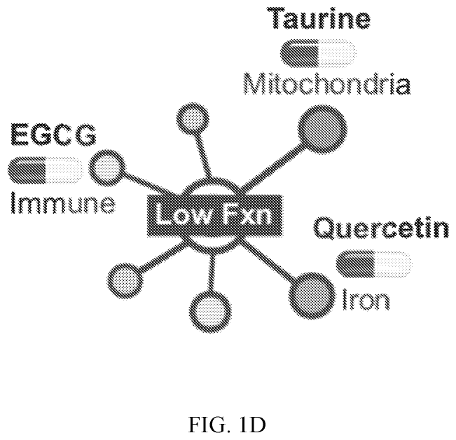

Using systems analyses in this mouse model after FXN knockdown revealed dysregulation of three stressor pathways related to cellular dysfunction. We found the immune, mitochondria, and iron stress pathways to be altered due to FXN deficiency (FIG. 1A).

Utilizing systems genomic computational analyses, three drugs were identified-epigallocatechin gallate (EGCG), taurine, and quercetin—that target the immune, mitochondria, and iron metabolism pathways, which are dysregulated in FRDA (FIG. 1D). We recently showed that immune system activation is among the earliest pathways regulated after FXN knockdown (Chandran V et al. 2017). Others and we have demonstrated that mitochondria and iron metabolism pathways are dysregulated after FA knockdown (Chandran V et al. 2017). Based on our systems analyses: (1) EGCG was predicted to modulate immune activation-responsive genes upregulated due to FXN reduction, (2) taurine was predicted to attenuate mitochondrial dysfunction and rescue mitochondria-related metabolic impairments due to FXN deficiency, and (3) quercetin was predicted to have multiple effects on iron homeostasis genes to reduce iron overload and increase FXN and NRF2 expression levels in vivo. Previous studies showed that quercetin caused serum and tissue iron depletion (Lesjak M et al. “Quercetin inhibits intestinal non-haem iron absorption by regulating iron metabolism genes in the tissues.” Eur. J. Nutrition 58:743-753 (2019)).

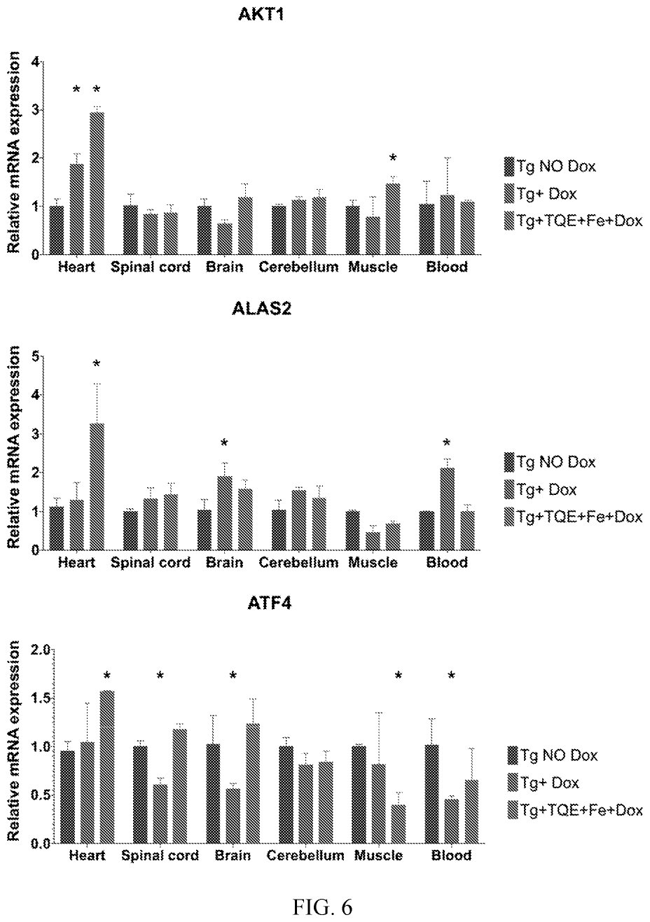

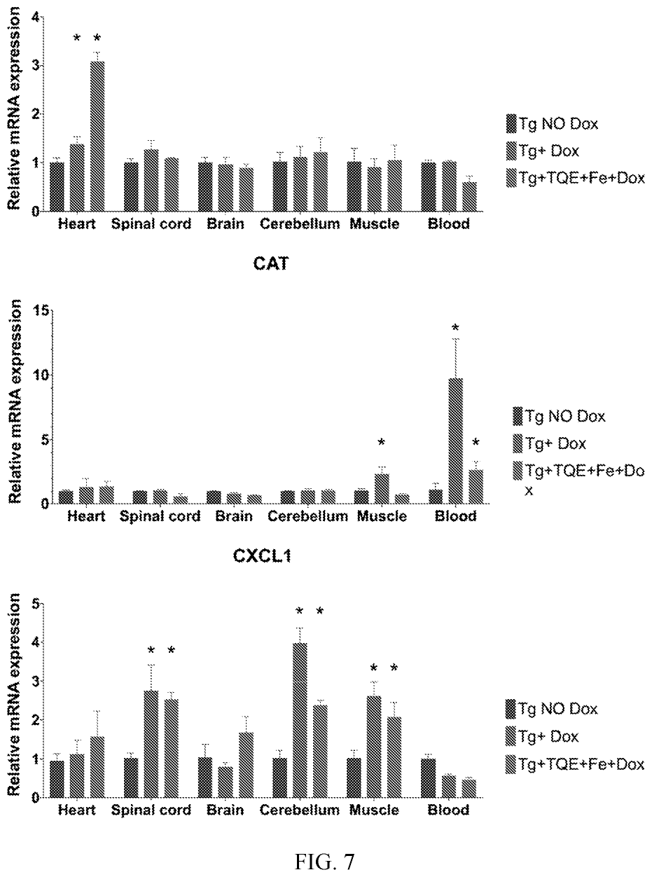

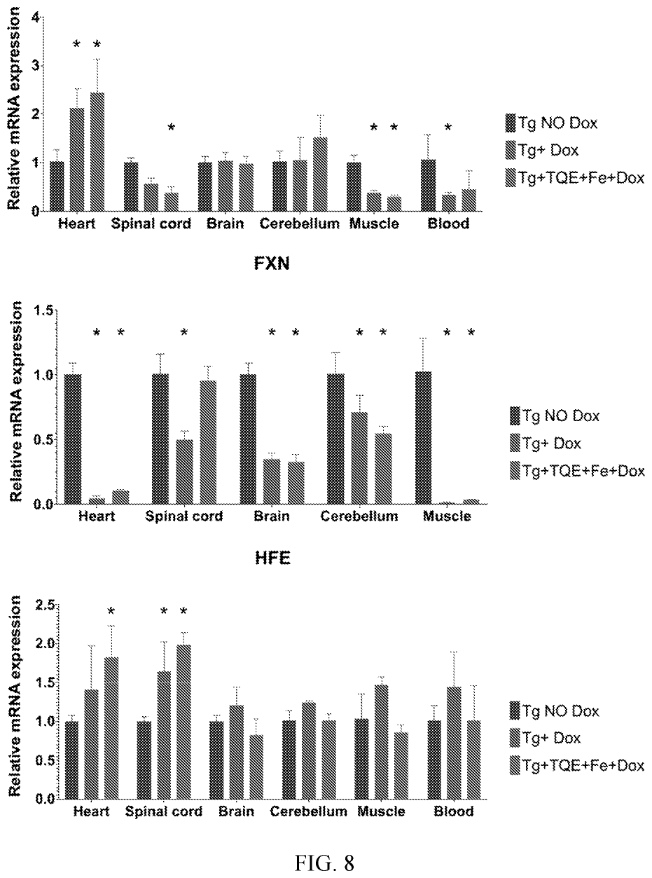

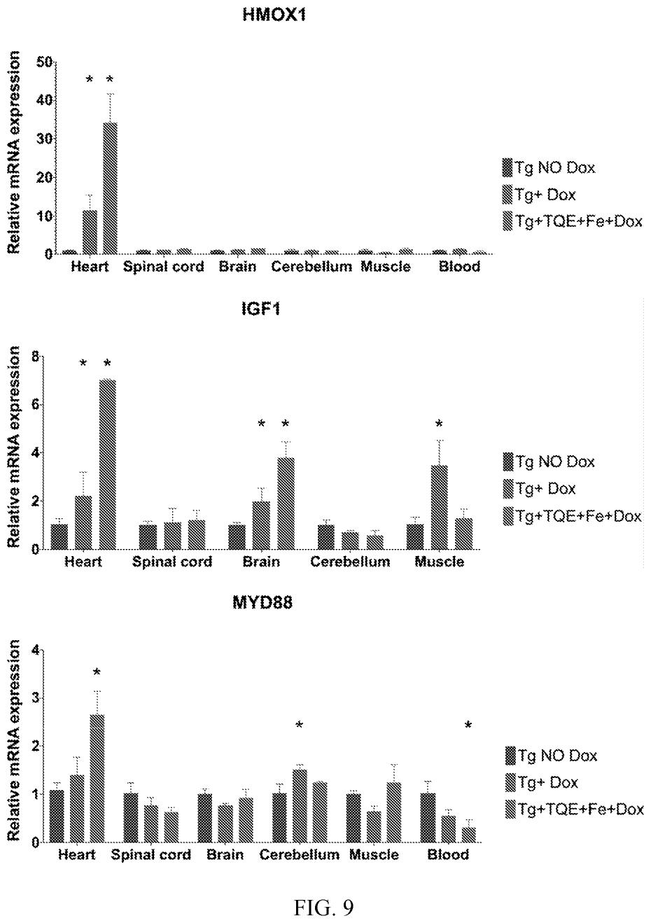

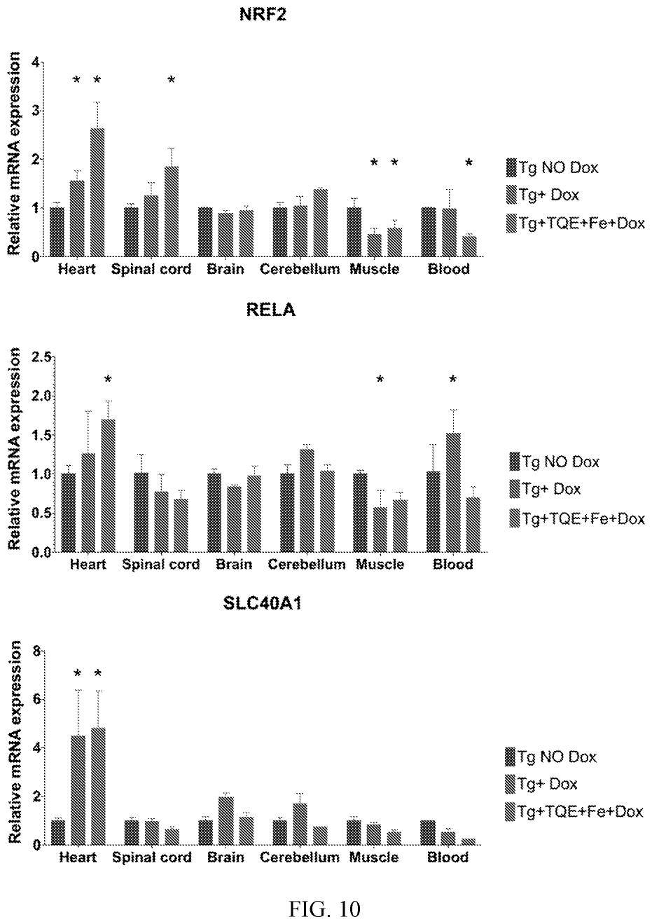

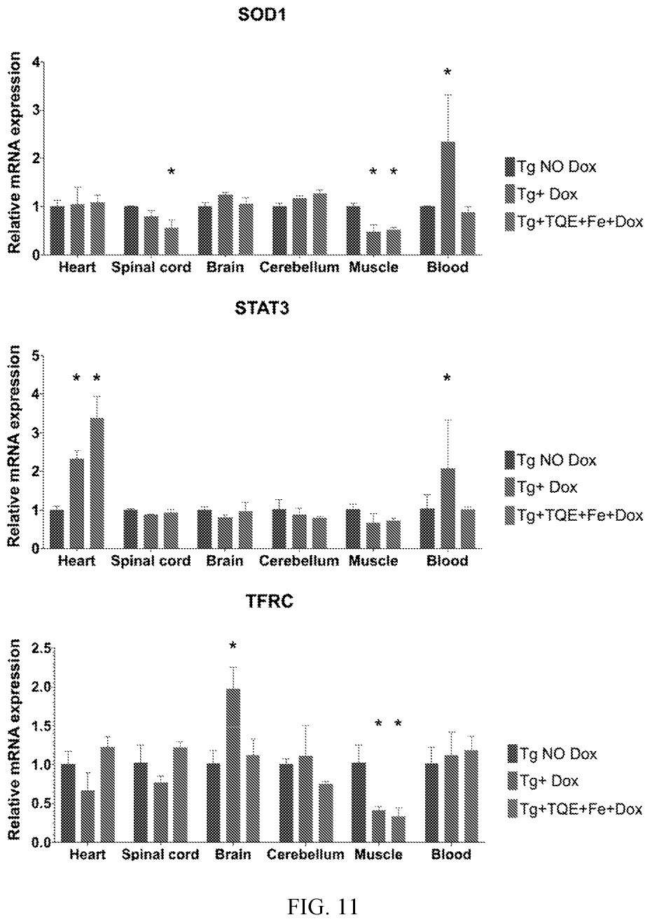

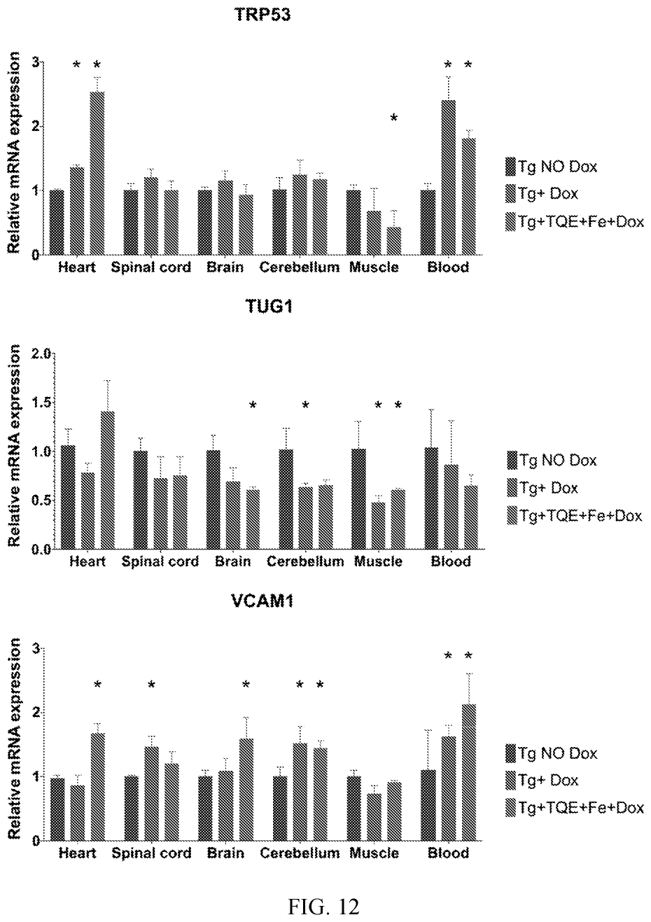

Combinatorial administration of EGCG, taurine, quercetin and ferrous sulfate to a FRDAkd mouse model demonstrated gene expression changes in various tissues. (FIGS. 6-12).

### Example 2. Combination Therapy in Treating FRDA

Combinations of EGCG, taurine, and quercetin, with or without ferrous sulfate, were assessed for efficacy in treating FRDA. The abilities of the combinations to increase endogenous FXN levels and modulate critical functional pathways altered due to FXN reduction in ameliorating or reversing FRDA-associated deficits were assessed.

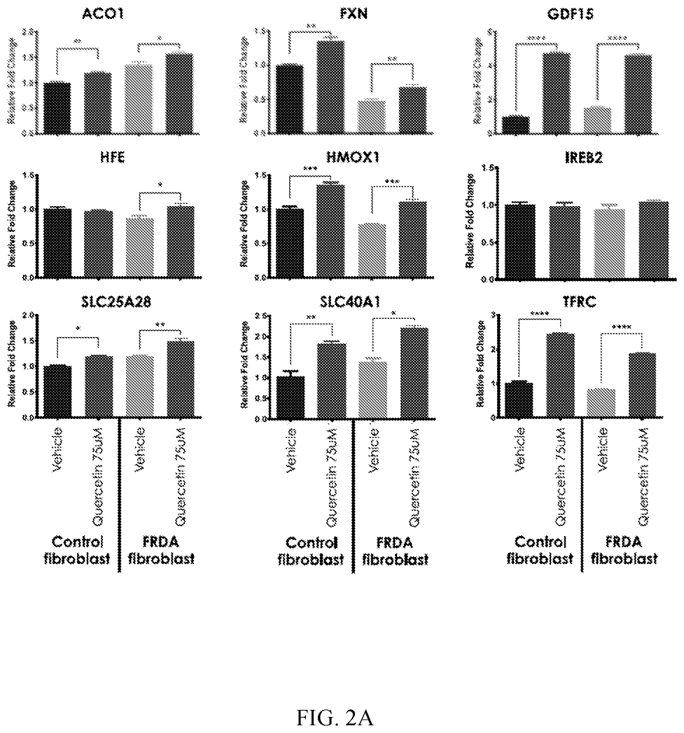

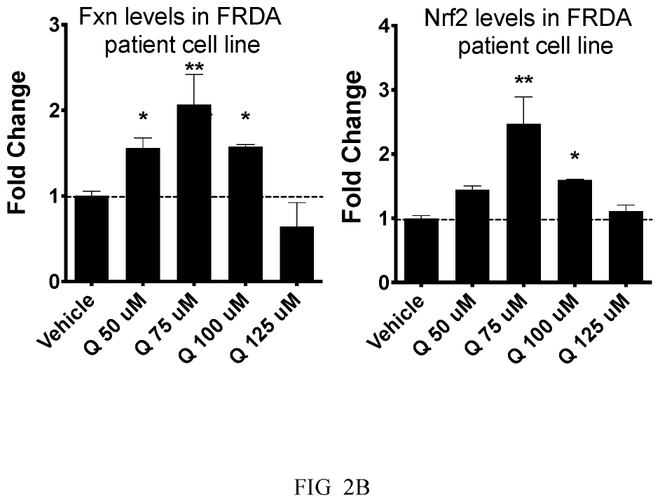

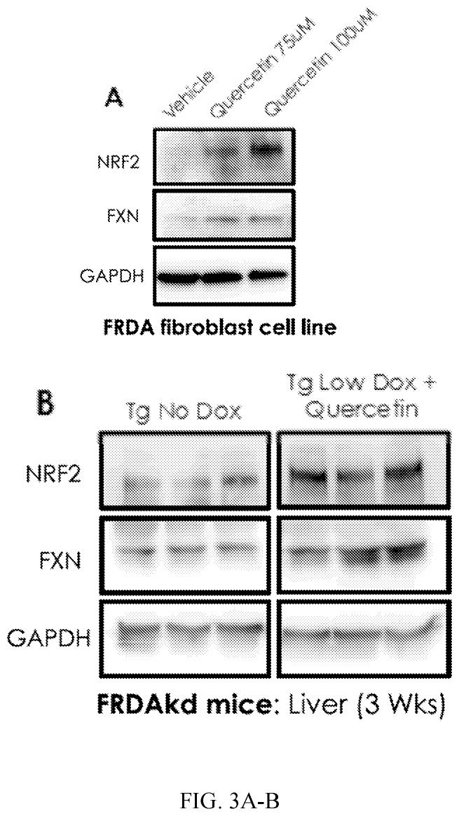

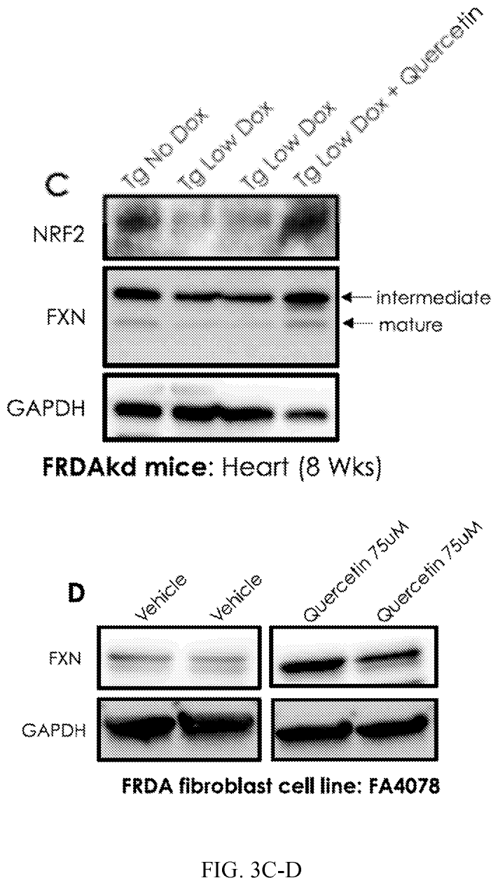

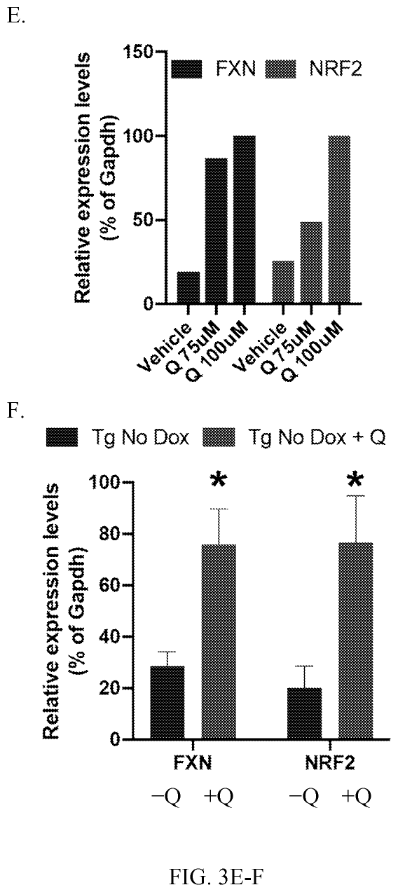

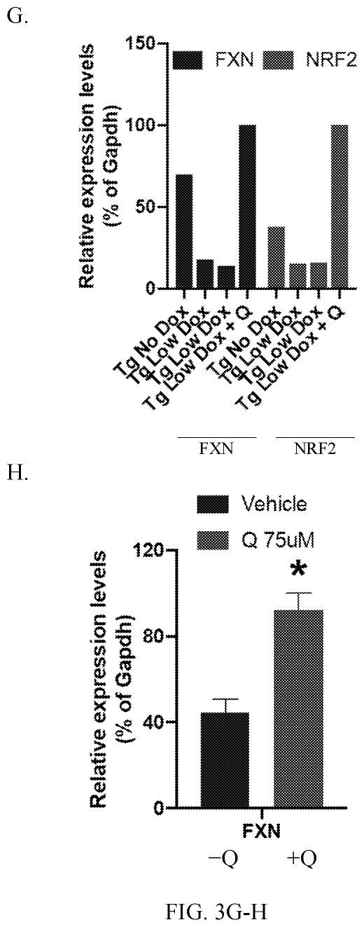

Quercetin administration was found to increase expression of several genes involved in iron metabolism in various cell lines, including the FRDA patient fibroblast cell lines (FIG. 2, 3). Quercetin administration was observed to increase FXN and NRF2 expression in FRDA patient fibroblast cell lines and in several tissues obtained from quercetin-treated FRDAkd mice (FIG. 3). FRDAkd mice are genetically engineered to knockdown expression of FXN in response of doxycycline (DOX) administration. DOX treated FRDAkd mice serve as a model of FRDA.

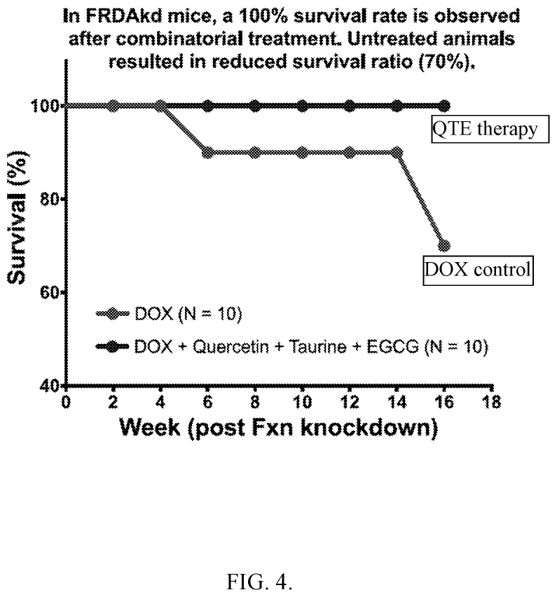

Combination therapy with taurine (100 mg/kg/day), quercetin (35 mg/kg/day), and EGCG (4.6 mg/kg/day), either with or without low dose ferrous sulfate (0.5 mg/kg/day), resulted in improvement in survival rate for FXN knockdown animals. 100% survival rate was observed in FRDAkd mice at 16 weeks post induction of FXN knockdown (DOX treatment) after combinatorial treatment (FIG. 4). In contrast, in the absence of the combination therapy, 30% mortality was observed in FRDAkd mice at 16 weeks post induction of FXN knockdown.

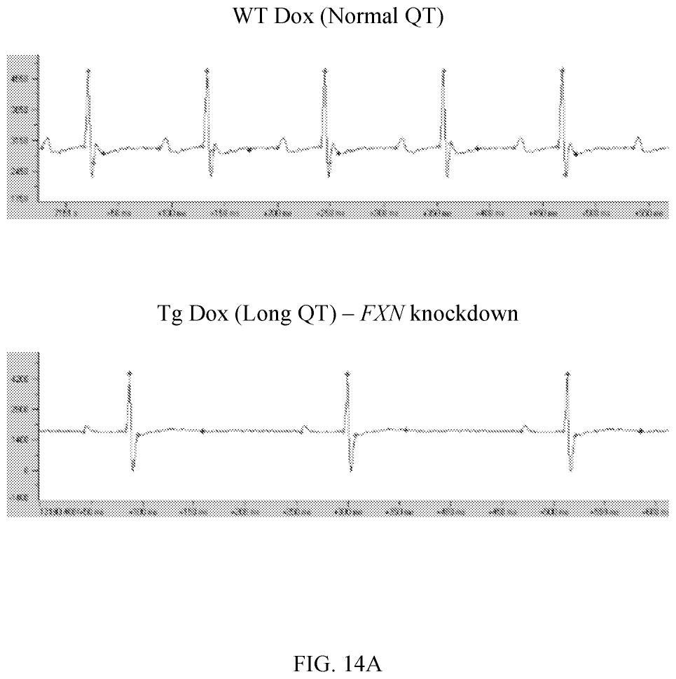

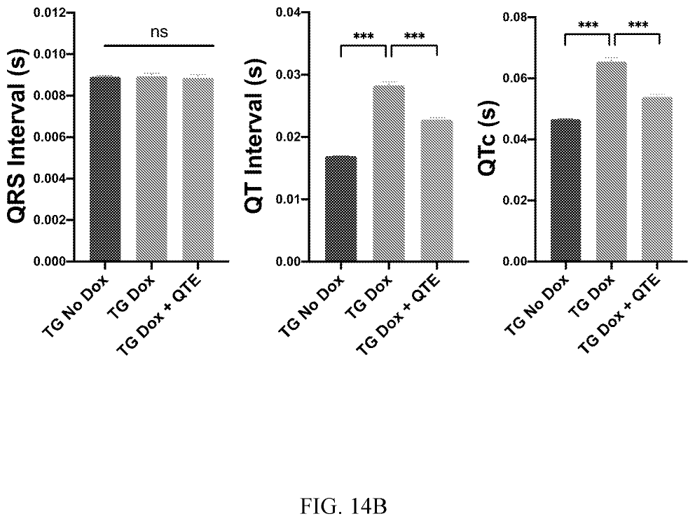

Electrocardiogram (ECG) data in FRDAkd mice treated with taurine, quercetin, and EGCG demonstrated a reversal of long QT intervals, suggesting that taurine/quercetin/EGCG therapy improved cardiac function (FIG. 14A).

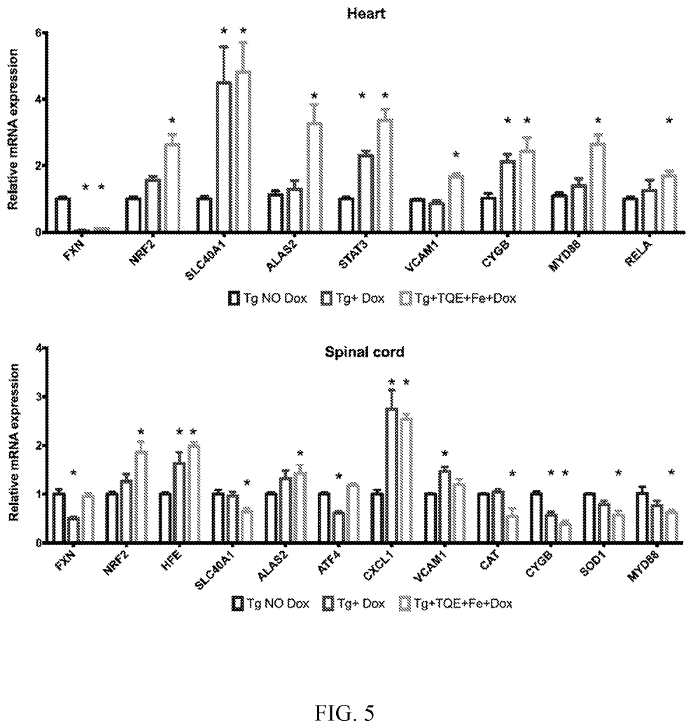

The combinatorial treatment also significantly modulated several FRDA-related genes, including Nrf2, in multiple tissues in vivo (FIGS. 5-12).

Using systems genomic computational analyses, we have identified 3 drugs (quercetin, taurine, and EGCG), modulating critical pathway genes associated with FRDA. Quercetin enhanced several genes including, FXN and NRF2 expression in vitro and in vivo, including in FRDA patient fibroblasts.

### Example 3. QTE Therapy Modulates Several FRDA-Associated Marker Genes

FXN knockdown (FRDAkd) animals were left untreated or treated with a combination of quercetin (35 mg/kg/day), taurine (100 mg/kg/day), and EGCG (4.6 mg/kg/day) (QTE therapy).

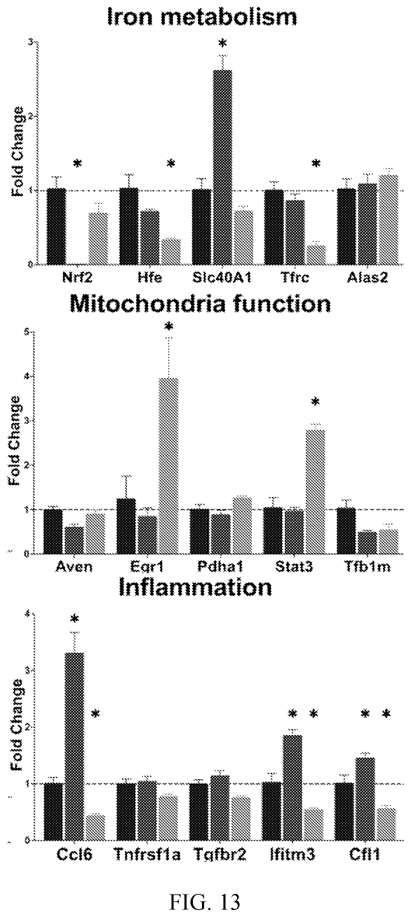

FRDAkd mouse hearts showed modulation of several critical genes under FXN-deficient conditions (FIG. 13). After 6 weeks of Dox (Fxn KD), significant NRF2 downregulation was observed in untreated animals. The NRF2 downregulation was reversed in animal treated with QTE therapy.

Iron overload was observed in untreated animals. Expression of genes involved in iron regulation (HFE), iron export (SLC40A1), and iron uptake (TFRC) were significantly downregulated in animals treated with QTE therapy (FIG. 13).

Transcriptional regulators EGR1 and STAT3 are known to improve mitochondrial function. Expression of these genes was elevated in animals treated with QTE therapy.

In additional several inflammation-related genes (CCL6, IFITM3, and CFL1) were downregulated in animal treated with QTE therapy.

Each of these effect of QTE therapy is consistent with QTE therapy alleviating symptoms associated with FRDA (FIG. 13).

### Example 4. QTE Therapy Improves Cardiac Function

FRDAkd mice displayed cardiomyopathy. Cardiac dysfunction is the most common cause of mortality in FRDA.

FXN knockdown (FRDAkd) animals were left untreated or treated with a combination of quercetin (35 mg/kg/day), taurine (100 mg/kg/day), and EGCG (4.6 mg/kg/day) (QTE therapy).

ECG in untreated FRDAkd mice revealed significantly increased QT interval durations compared with control (no DOX) groups at 12 and 24 weeks, suggesting abnormal heart rate and arrhythmia (FIG. 14A). Similar cardiac abnormalities have been observed in clinical settings. FRDAkd mice treated with QTE therapy exhibited a reversal of the long QT interval, suggesting that QTE therapy improves cardiac function (FIG. 14B).

1. A composition for treating Friedreich's ataxia (FRDA) comprising two or more of quercetin, taurine, epigallocatechin gallate, and ferrous sulfate.

2. The composition of claim 1, wherein two or more comprises three or more.

3. The composition of claim 2, wherein three or more comprises:
(a) quercetin, taurine, and epigallocatechin gallate;
(b) quercetin, taurine, and ferrous sulfate;
(c) quercetin, epigallocatechin gallate, and ferrous sulfate; or
(d) taurine, epigallocatechin gallate, and ferrous sulfate.

4. The composition of claim 3 wherein the composition comprises quercetin, taurine, and epigallocatechin gallate.

5. The composition of claim 4, wherein the composition further comprises ferrous sulfate.

6. The composition of any one of claims 1-5, further comprising a pharmaceutically acceptable excipient.

7. The composition of any one of claims 1-6, wherein the composition is for formulated for enteral administration, oral administration, parenteral administration, intravascular administration, intravenous administration, rectal administration, intraperitoneal injection, subcutaneous injection, transcutaneous administration, or intramuscular injection.

8. The composition of any one of claims 1-7, wherein the composition is formulated as a liquid, an aqueous solution, a suspension, a solid, a powder, a granule, or a pill.

9. The composition of claim 8, wherein the pill comprises a lozenge, a capsule, a tablet, or a caplet.

10. The composition of any one of claims 1-9, wherein the composition is formulated for repeat dosing.

11. A method of treating FRDA comprising administering to a subject having FRDA, diagnosed with FRDA, or at risk of developing FRDA the composition of any one of claims 1-10.

12. The method of claim 11, wherein the subject has loss of function mutations in both copies of their FXN genes, where in the loss of function mutations are independently selected from the group consisting of can be a GAA trinucleotide repeat expansion within the first intron of the FXN gene, a nonsense mutation, a frame shift mutation, insertion, deletion, or missense mutation that reduces function of the encoded frataxin.

13. The method of claim 11 or 12, wherein the composition is administered to the subject prior to appearance of one or more symptoms associated with FRDA.

14. The method of claim 11 or 12, wherein the composition is administered to the subject subsequent to appearance of one or more symptoms associated with FRDA.

15. The method of claim 13, wherein administering the composition to the subject prevents or delays onset of the one or more symptoms associated with FRDA.

16. The method of claim 14, wherein administering the composition to the subject reduces the severity or delays progression of the one or more symptoms associated with FRDA.

17. The method of any one of claims 13-16, wherein the one or more symptoms associated with FRDA are selected from the group consisting of: gait ataxia, difficulty walking, poor balance, loss of sensation in the arms and legs, slowness and slurring of speech (dysarthria), hesitant and jerky speech, difficulty coordinating movement (ataxia), muscle weakness, spasticity, scoliosis, difficulty swallowing, hearing loss, vision loss, heart disease, heart palpitations, shortness of breath, hypertrophic cardiomyopathy, myocardial fibrosis, heart rhythm abnormalities, tachycardia, and heart block.

18. The method of any one of claims 11-17, wherein treating the subject increases survival of the subject.

19. A method for modulating expression of one or more genes dysregulated in Friedreich's ataxia in a subject comprising administering to the subject having FRDA, diagnosed with FRDA, or at risk of developing FRDA, the composition of any one of claims 1-10.

20. The method of claim 19, wherein the one or more genes is selected from the group consisting of: ACO1, AKT1, ALAS2, ATF4, AVEN, BIRC5, CASP8, CAT, CCL6, CFL1, CXCL1, CYGB, EGR1, FXN, GDF15, HFE, HMOX1, IFITM3, IGF1, IREB2, MMP2, MYD88, NRF2, PDHA1, RELA, SLC25A28, SLC40A1, SOD1, STAT3, TFB1M, TFRC, TGFBR2, TNFRSF1A, TRP53, TUG1, and VCAM1.

21. A kit for treating FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the kit comprising the composition of any one of claims 1-9 and instructions for use.

22. A composition for treating or preventing Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, or spinocerebellar ataxia, the composition comprising quercetin, taurine, and epigallocatechin gallate.

23. A method for treating a subject suffering from or at risk of developing a disease characterized in involving iron metabolism, mitochondrial dysfunction, and immune pathways, the method comprising administering to the subject a first compound that regulates iron metabolism, a second compound that reduces mitochondrial dysfunction, and a third compound that modulates immune function.

24. The method of claim 23, wherein the disease is selected from the group consisting of: FRDA, Alzheimer's disease, amyotrophic lateral sclerosis, ataxia, autism spectrum disorder, cerebellar ataxia, fragile X syndrome, frontotemporal dementia, Huntington's disease, Lewy body disease, mitochondrial cardiomyopathy, motor neuron disease, multiple sclerosis, multiple system atrophy, muscular dystrophy, neurodegeneration with brain iron accumulation, Parkinson's disease, progressive supranuclear palsy, spinal muscular atrophy, and spinocerebellar ataxia.

25. The method of claims 23 or 24, wherein the first, second and third compounds comprise: quercetin, taurine, and epigallocatechin gallate.

26. A method of increasing FXN expression in a subject having FRDA, diagnosed with FRDA, or at risk of developing FRDA, comprising administering to the subject the composition of any one of claims 1-10.

27. A method of increasing iron metabolism, reducing mitochondrial dysfunction, and modulating immune function in a subject comprising administering to the subject the composition of any one of claims 1-10.
